(19) United States
(12) Patent Application Publication
(10) Pub. No.: US 2022/0235326 A1
(43) Pub. Date: Jul. 28, 2022

(54) ENHANCEMENT OF FIBROBLAST THERAPEUTIC ACTIVITY BY RNA

(71) Applicant: Figene, LLC, Houston, TX (US)

(72) Inventors: Pete O'Heeron, Houston, TX (US); Thomas lchim, San Diego, CA (US)

(21) Appl. No.: 17/596,336

(22) PCT Filed: Jun. 12, 2020
(86) PCT No.: PCT/US2020/037467

§ 371 Date: Dec. 8, 2021

Related U.S. Application Data
(60) Provisional application No. 62/860,252, filed on Jun. 12, 2019.

(51) Int. Cl. C12N 5/077; A61K 35/33

(52) U.S. Cl. CPC C12N 5/0656; A61K 35/33; C12N 2533/32; C12N 2500/40; C12N 2501/90; C12N 2501/12

(57) ABSTRACT

Embodiments of the disclosure encompass methods and compositions related to the ability of RNA to enhance therapeutic activity of fibroblasts. In some embodiments, administration of double stranded RNA is performed through providing polyinosinicpolycytidylic acid (poly (I:C)) or a derivative thereof at a concentration sufficient to induce therapeutic properties and/or to augment therapeutic properties onto said fibroblasts. In one embodiment, enhanced therapeutic activity comprises augmentation of fibroblast migratory activity; efficacy for angiogenesis; efficacy for immune modulation; differentiation ability; production of one or more trophic factors; and/or the ability to resist apoptosis.

This application claims priority to U.S. Provisional Patent Application Ser. No. 62/860,252, filed Jun. 12, 2019, which is incorporated by reference herein in its entirety.

### TECHNICAL FIELD

The technical field of the disclosure includes at least the fields of cell biology, molecular biology, cell therapy, and medicine.

### BACKGROUND

Fibroblasts comprise the main cell type of connective tissue, possessing a spindle-shaped morphology, whose classical function has historically been believed to produce extracellular matrix responsible for maintaining the structural integrity of tissue. Fibroblasts also play an important role in proliferative phases of wound healing, resulting in deposition of extracellular matrix [1, 2]. During wound healing, scar tissue is formed by fibroblast over proliferation. In embryos, and in some types of amphibians, scar-less healing occurs after injury by processes which are currently under intense investigation [3, 4]. With aging, many kinds of tissues and organs undergo fibrosis gradually, such as fibrosis of skin, lung, liver, kidney and heart. The process of scar tissue formation is caused by hyperproliferation of fibroblasts, as well as these cells producing abnormally large amounts of extracellular matrix and collagens during proliferation and thereby replacing normal organ structure (parenchyma), leading to functional impairment and scar formation, which may further trigger persistent fibrosis.

The present disclosure provides solutions to long felt needs in the art of providing therapeutic compositions for cell therapy.

### BRIEF SUMMARY

Embodiments of the disclosure include methods and compositions for augmentation of fibroblast cell transplantation efficacy. More specifically, in specific embodiments the disclosure there are means of generating fibroblast cells possessing enhanced therapeutic activity after transplantation. In specific embodiments, the disclosure pertains to means of utilizing exposure to an effective amount of RNA as a method of increasing therapeutic activity of fibroblasts.

The current disclosure provides compositions of matter, treatment protocols, and methods of use based on the previously unknown ability of RNA to enhance therapeutic activity of fibroblasts. In some embodiments, administration of double stranded RNA is performed through providing polyinosinic-polycytidylic acid (poly (I:C)) or a functionally active derivative thereof at a concentration sufficient to induce one or more therapeutic properties and/or to augment therapeutic properties onto the fibroblasts. In one embodiment, enhanced therapeutic activity comprises augmentation of fibroblast migratory activity. In other embodiments, therapeutic activities are selected from a group comprising of: a) angiogenesis; b) immune modulation; c) differentiation ability; d) production of trophic factors; e) ability to resist apoptosis; f) migratory activity; and g) a combination thereof.

It is specifically contemplated that any limitation discussed with respect to one embodiment of the invention may apply to any other embodiment of the disclosure. Furthermore, any composition of the invention may be used in any method of the disclosure, and any method of the disclosure may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects and advantages of the present invention will be set forth in part in the description which follows, and in part will be obvious from the description or may be learned by practice of the invention. Various embodiments of the disclosure will be described in sufficient detail to enable those skilled in the art to practice the invention, and it is to be understood that other embodiments may be utilized and that changes may be made without departing from the scope of the invention. The following detailed description is, therefore, not be taken in a limiting sense, and the scope of the present invention is best defined by the appended claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIG. 1 provides an example of a cell migration assay. Triangles refer tp Poly I:C, “X” refers to CpB, and squares are scrambled RNA; and

FIG. 2 shows HGF production from fibroblasts following exposure to Poly (I:C). Bars from left to right are Control, low molecular weight Poly (I:C), and high molecular weight Poly (I:C).

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “exogenous” as used herein refers to RNA that originated from outside the fibroblast cells.

The term “subject,” as used herein, which may be used interchangeably with the term “patient” or “individual,” generally refers to an individual having a need to treat or prevent a medical condition that utilizes cell therapy and particularly fibroblast therapy. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, one or more chronic medical conditions, one or more injuries, or any combination thereof. The disease may or may not be pathogenic. The subject may being undergoing or having undergone antibiotic treatment. The subject may be asymptomatic. The subject may be healthy individuals. The “subject” or “individual”, as used herein, may or may not be housed in a medical facility and may be treated as an outpatient of a medical facility. The individual may be receiving one or more medical compositions via the internet. An individual may comprise any age and any gender of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

Embodiments of the disclosure include methods of enhancing one or more therapeutic activities of a fibroblast population comprising the steps of: a) optionally selecting a fibroblast population; and b) treating a fibroblast population with a concentration of RNA sufficient to enhance one or more therapeutic properties of the fibroblasts. The RNA may or may not be double stranded RNA, such as double stranded RNA being polyinosine-polycytidylic acid (Poly (I:C)). The double stranded RNA may be polyinosine-polycytidylic acid stabilized with Polylysine and Carboxymethylcellulose (Poly ICLC). In specific cases, the therapeutic activity of the fibroblasts comprises production of one or more angiogenic factors. The therapeutic activity of the fibroblasts may comprise production of one or more regenerative factors. The therapeutic activity of the fibroblasts may comprise migratory activity towards injury associated signals. The therapeutic activity of the fibroblasts may comprise reduction of apoptosis. The therapeutic activity of the fibroblasts may comprise any of these in combination.

The fibroblasts may be derived from a source selected from the group consisting of a) adipose tissue; b) dermal tissue; c) placental tissue; d) hair follicles; e) keloid tissue; f) bone marrow; g) peripheral blood; h) umbilical cord; i) foreskin; and j) a combination thereof.

Pharmaceutical preparations of cells may comprise any of the fibroblasts encompassed herein. The preparations may or may not also comprise RNA, such as Poly (I:C).

Methods of producing the fibroblast cells are encompassed herein. In specific embodiments, any method may or may not include the step of inducing activation of toll like receptor 3, for example through contact with a ligand capable of inducing an interferon response in the fibroblast cells. The method may further comprise the step of delivering a therapeutically effective amount of the cells to an individual at risk of having a medical condition or that has a medical condition for which the cells would be therapeutic, such as remove or reduce the severity of at least one symptom.

The disclosure discloses the previously unknown and paradoxical properties of the use of RNA molecules to enhance therapeutic activity of fibroblast cells. In one embodiment, RNA molecules are utilized in a sequence non-specific, and/or sequence semi-specific manner in order to activate molecular pathways inside fibroblasts capable of inducing production of one or more interferons. In one embodiment, the disclosure provides that the production of one or more interferons is associated with enhanced ability of the fibroblasts to migrate towards an area of injury, for example, towards a SDF-1 gradient that could be associated with an injury. In other embodiments the disclosure additionally or alternatively provides the stimulation of fibroblasts with one or more activators of the PKR pathway, including toll like receptor 3, in order to enhance therapeutic activity, wherein the therapeutic activity includes enhancement of migration, augmented production of cytokines, and/or elevated ability to stimulate production of new blood vessels (angiogenesis, arteriogenesis and/or vasculogenesis).

In one embodiment, the disclosure provides the use of Poly (I:C) as a source of double stranded RNA to stimulate and/or enhance therapeutic properties of fibroblasts. Without being bound to theory, Poly (I:C) in specific embodiments possesses the property of Toll Like Receptor 3 (TLR3) ligand that mimics viral RNA and is a known stimulant of the innate immune response. When Poly (I:C) contacts fibroblasts, expression of anti-viral proteins like Interferon alpha and/or beta are induced. For the purpose of the disclosure, Poly (I:C) is a synthetic double-stranded RNA comprised of anti-parallel polynucleotide strands of inosinic acid and cytidylic acid sodium salts. The strands are non-covalently bound by hydrogen bonds formed between the inosine and cytosine bases. The average chain length for the Poly (I:C) ranges between 300 to 6,000 base pairs, corresponding to approximately 180,000 to 3,600,000 daltons. The molecular formula is (C₁₀H₁₀N₄NaO₇P)ₓ.(C₉H₁₁NaN₃O₇P)ₓ. In some embodiments, given that Poly (I:C) is an unstable molecule in aqueous solutions, to achieve an effective fibroblast augmentation effect, Poly (I:C) is re-dissolved immediately prior to use and in some situations multiple in vitro administrations may be required. In some embodiments, poly (I:C) may be formulated with one or several bioadhesive polymers that can prolong the residence time in tissue culture, in order to maintain fibroblast activation.

In some embodiments, the disclosure relates to a composition of fibroblasts that have been activating using micro particles of polyinosinic-polycytidylic acid (Poly (I:C)) and a carrier polymer selected from starch, alginate, blanose or DPPC (dipalmitoylphosphatidylcholine). Micro particles are particles with an average particle size between 0.1 μm and 100 μm. In particular cases, the carrier polymer is starch, such as obtained from maize, potato or cassava. In other embodiments, nanoparticles may be utilized for delivery of Poly (I:C) to fibroblasts in vitro. In some embodiments, admixture of poly (I:C) and starch is performed, The ratio Poly (I:C)/starch according to the disclosure ranges from 1/200 (w/w) to 1/0.1 (w/w), but particularly from 1/100 (w/w) to 1/1 (w/w) and even more preferably from 1/100 (w/w) to 1/5 (w/w) while a ratio Poly (I:C)/starch between 1/12 and 1/9 (w/w) may be utilized.

The fibroblast cells may be cultured fibroblasts cells, and when referring to cultured fibroblast cells, the term senescence (also replicative senescence or cellular senescence) refers to a property attributable to finite cell cultures; namely, their inability to grow beyond a finite number of population doublings (sometimes referred to as Hayflick's limit). The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. As used herein, the term Growth Medium generally refers to a medium sufficient for the culturing of umbilicus-derived cells. In particular, one particular medium for the culturing of the cells of the disclosure herein comprises Dulbecco's Modified Essential Media (also abbreviated DMEM herein). Particularly considered is DMEM-low glucose (also DMEM-LG herein) (Invitrogen, Carlsbad, Calif.). The DMEM-low glucose may be supplemented with 15% (v/v) fetal bovine serum (e.g., defined fetal bovine serum, Hyclone, Logan Utah), antibiotics/antimycotics (such as penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

The fibroblast cells may be cultured in standard growth conditions. Also relating to the present disclosure, the term standard growth conditions, as used herein refers to culturing of cells at 37° C., in a standard atmosphere comprising 5% CO₂. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In one embodiment of the disclosure, fibroblasts treated with RNA are utilized to treat one or more inflammatory conditions. In such cases, the term “inflammatory conditions” includes, for example: (1) tissue damage due to ischemia-reperfusion following acute myocardial infarction, aneurysm, stroke, hemorrhagic shock, crush injury, multiple organ failure, hypovolemic shock intestinal ischemia, spinal cord injury, and traumatic brain injury; (2) inflammatory disorders, e.g., burns, endotoxemia and septic shock, adult respiratory distress syndrome, cardiopulmonary bypass, hemodialysis; anaphylactic shock, severe asthma, angioedema, Crohn's disease, sickle cell anemia, poststreptococcal glomerulonephritis, membranous nephritis, and pancreatitis; (3) transplant rejection, e.g., hyperacute xenograft rejection; (4) pregnancy related diseases such as recurrent fetal loss and pre-eclampsia, and (5) adverse drug reactions, e.g., drug allergy, IL-2 induced vascular leakage syndrome and radiographic contrast media allergy. Complement-mediated inflammation associated with autoimmune disorders including, but not limited to, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barre syndrome, Hashimoto's disease, idiopathic thrombocytopenic purpura, pemphigus, Sjogren's syndrome, and Takayasu's arteritis, may also be treated with the methods described herein.

In some embodiments of the disclosure, fibroblasts treated with RNA are used to treat one or more neurodegenerative conditions. A “neurodegenerative condition” (or disorder) encompasses acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, e.g. due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

The fibroblasts for use in any method of the current disclosure may be of any mammalian origin, e.g., human, rat, primate, porcine and the like. In one embodiment of the disclosure, the fibroblasts are derived from human umbilicus. Umbilicus-derived cells are capable of self-renewal and expansion in culture, and have the potential to differentiate into cells of other phenotypes. Methods of deriving cord tissue fibroblast cells from human umbilical tissue are contemplated. The cells are capable of self-renewal and expansion in culture, and have the potential to differentiate into cells of other phenotypes. The method comprises one or more steps of (a) obtaining human umbilical tissue; (b) removing substantially all of blood to yield a substantially blood-free umbilical tissue, (c) dissociating the tissue by mechanical or enzymatic treatment, or both, (d) re-suspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment. Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In one embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g., deoxyribonucleases and the neutral protease, dispase) to strongly digestive (e.g., papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently considered are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion may be serum-free. EDTA and DNase are commonly used and may improve yields or efficiencies. Particular methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. Particular methods include those methods that employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIB ERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Certain enzyme treatments may be 0.5, 1, 1.5, or 2 hours long or longer. In other particular embodiments, the tissue is incubated at 37° C. during the enzyme treatment of the dissociation step. Diluting the digest may also improve yields of cells as cells may be trapped within a viscous digest.

While the use of enzyme activities is utilized in some embodiments, it is not required for isolation methods as provided herein. Methods based on mechanical separation alone may be successful in isolating the instant cells from the umbilicus as discussed above.

The cells can be re-suspended after the tissue is dissociated into any culture medium as discussed herein above. Cells may be re-suspended following a centrifugation step to separate out the cells from tissue or other debris. Resuspension may involve mechanical methods of re-suspending, or simply the addition of culture medium to the cells.

Providing the growth conditions allows for a wide range of options as to culture medium, supplements, atmospheric conditions, and relative humidity for the cells. A particular temperature is 37° C., however the temperature may range from about 35° C. to 39° C. depending on the other culture conditions and desired use of the cells or culture.

In some embodiments, presently considered are methods that provide cells that require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately re-suspended and grown in. In this sense, the method is selective for those cells capable of division in the absence of the particular growth factors. Particular cells in some embodiments are capable of growth and expansion in chemically-defined growth media with no serum added. In such cases, the cells may require certain growth factors, which can be added to the medium to support and sustain the cells. In specific embodiments, factors to be added for growth on serum-free media include one or more of FGF, EGF, IGF, and PDGF. In some embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10¹⁴ cells or more are provided. Preferred are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, cord tissue fibroblast cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR, DP, DQ.

In some embodiments, fibroblasts are collected from donors and information about each donation is recorded. In some specific embodiments, the recorded information comprises at least some data selected from the group consisting of the type of cells, their tissue of origin, the date of their collection and the identity of the donor. In other specific embodiments, the recorded information comprises results obtained from various characterization assays. Examples include HLA typing, determining the presence of specific markers, determining specific SNP alleles and/or performing a nucleated cell count on the stem cell unit. In some embodiments, the collected cells are sorted according to at least one criterion. In some specific embodiments, they are sorted according to their type, their tissue of origin, the date of their collection and the donor identity. Particular populations of fibroblasts may be utilized based on the obtained information.

In some embodiments, the collected fibroblasts are stored under appropriate conditions to keep the cells viable and functional. In some specific embodiments, the fibroblasts are stored under cryopreservation conditions. In other embodiments, said fibroblasts are stored in the bank are for allogeneic use. In some embodiments, the stored stem cells are used for allogeneic transplantations. In other embodiments, the stored fibroblasts are used for the establishment of cell lines having, for example, good viability and other desirable characteristics for research and pharmaceutical applications.

In some embodiments, the fibroblasts stored in a depository such as a bank are arranged in units. According to these embodiments, each donation to the bank (each deposit of fibroblasts) is divided into a plurality of units. In some typical embodiments, a unit comprises a population of fibroblasts of the same type that were collected from a single donor in a single donation. In some exemplary embodiments, a unit includes fibroblasts expressing a specific marker or markers. In some embodiments, a unit is further defined by the number of nucleated cells present in the sample. Upon request, one or more units may be allocated to a subject in need thereof. In some embodiment, a fraction of a unit is allocated to a recipient in need. In some typical embodiments, the number of units to be allocated depends on the number of nucleated cells in each unit and the medical condition to be treated. In some embodiments, the amount of fibroblasts, or the number of units, available for allocation to an individual depends on the amount of donations made.

In some embodiments, the fibroblasts can be subjected to further processing after their collection. In some specific embodiments, the collected fibroblasts can be cultured, expanded and/or proliferated. In additional specific embodiments, the collected fibroblasts are processed in order to achieve therapeutic levels. In some embodiments, an optimal combination of fibroblasts can be selected from the reservoir of cells, in order to treat a certain pathological condition.

According to another aspect, the present disclosure provides a method of fibroblast banking, the method comprising periodically collecting a plurality of donations from an individual throughout the individual's life. In some embodiments, the method comprises collecting fibroblasts from more than one source. In some embodiments, the method comprises collecting fibroblasts of more than one type.

In some embodiments of the disclosure, donor cells are modulated to possess enhanced therapeutic properties.

In some embodiments of the disclosure, fibroblasts are transfected to possess enhanced neuromodulatory and neuroprotective properties. The transfection may be accomplished by use of any type of vector, including viral vectors or non-viral vectors. Viral vectors include lentiviral, adenoviral, retroviral, or adeno-associated viral vectors, as examples. In one embodiment, lentiviral vectors are utilized, and means to perform lentiviral mediated transfection are well-known in the art and discussed in the following references [5-11]. Some specific examples of lentiviral based transfection of genes into fibroblasts include transfection of SDF-1 to promote stem cell homing, particularly hematopoietic stem cells [12], GDNF to treat Parkinson's in an animal model [13], HGF to accelerate remyelination in a brain injury model [14], akt to protect against pathological cardiac remodeling and cardiomyocyte death [15], TRAIL to induce apoptosis of tumor cells [16-19], PGE-1 synthase for cardioprotection [20], NUR77 to enhance migration [21], BDNF to reduce ocular nerve damage in response to hypertension [22], HIF-1 alpha to stimulate osteogenesis [23], dominant negative CCL2 to reduce lung fibrosis [24], interferon beta to reduce tumor progression [25], HLA-G to enhance immune suppressive activity [26], hTERT to induce differentiation along the hepatocyte lineage [27], cytosine deaminase [28], OCT-4 to reduce senescence [29, 30], BAMBI to reduce TGF expression and protumor effects [31], HO-1 for radioprotection [32], LIGHT to induce antitumor activity [33], miR-126 to enhance angiogenesis [34, 35], bcl-2 to induce generation of nucleus pulposus cells [36], telomerase to induce neurogenesis [37], CXCR4 to accelerate hematopoietic recovery [38] and reduce unwanted immunity [39], wnt11 to promote regenerative cytokine production [40], and the HGF antagonist NK4 to reduce cancer [41].

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Enhanced Migration Towards Stromal Cell-Derived Factor 1 (SDF-1)

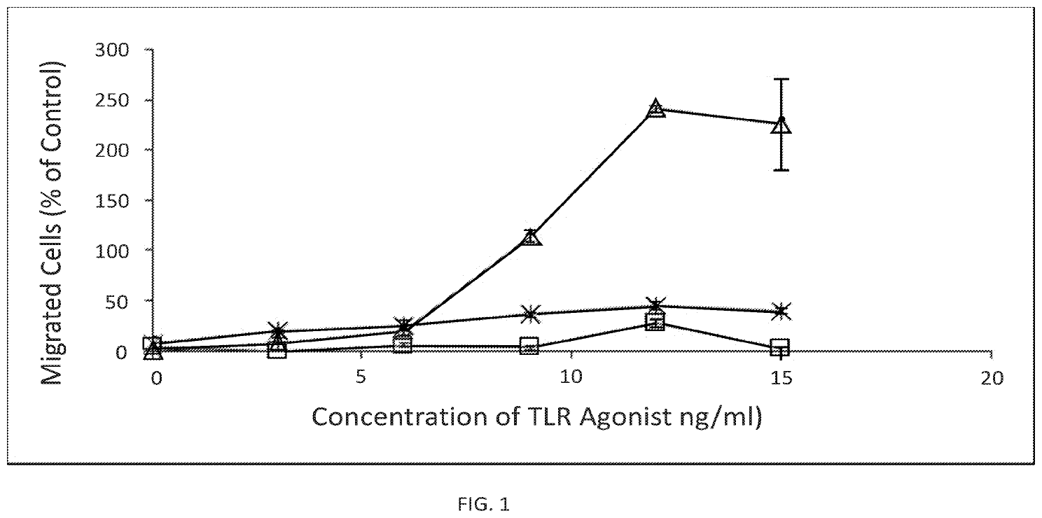

Cells were assessed for chemotaxis to the indicated chemokine (SDF-1) under normoxic conditions for 2 h (FIG. 1). Migrated cells were collected from the lower migration chamber compartments and counted. Cells were seeded at 2.5×10⁶/mL in the upper chamber of a Transwell system (3 mm pore size; Corning Costar, 3415). 10% FBS RPMI 1640 medium alone or supplemented with recombinant human CXCL12 (100 ng/mL) (Peprotech, 300-28A), or CCL19 (0.3 μg/mL), or CCL21 (0.6 μg/mL) was added to the lower compartment. Cells were allowed to migrate for 2 h at 37° C. under normoxic condition. Cells migrated in the lower chamber were collected and counted.

### Example 2

### Enhancement of Fibroblast Hepatocyte Growth Factor (HGF) Production from Fibroblasts by Poly (I:C)

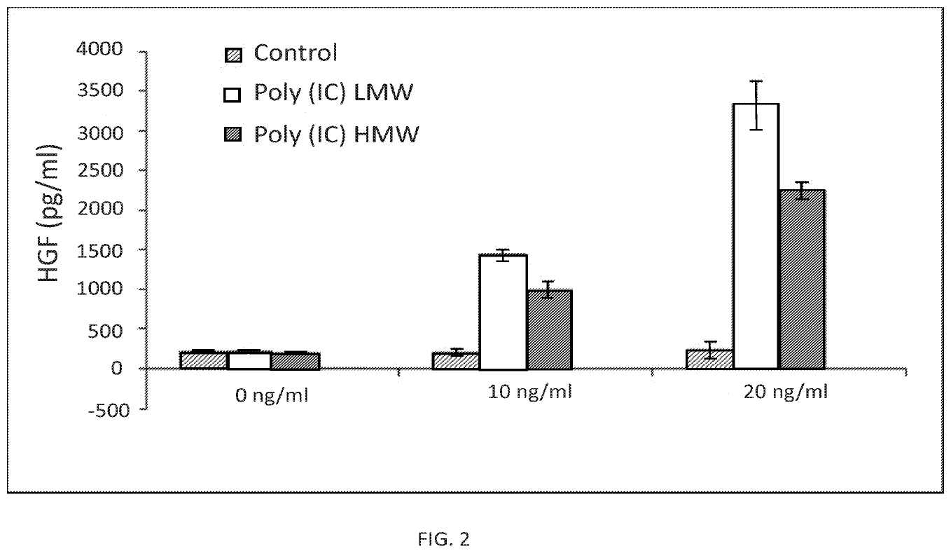

Fibroblasts were cultured as in Example 1 and treated with control, low, or high molecular weight Poly (I:C) from InvivoGen® (San Diego, Calif.). Cells were cultured for 48 hours and HGF concentration was assessed using ELISA. Substantial stimulation of HGF production was noted with both high and low molecular weight Poly (I:C) (FIG. 2). HGF is one example of a cytokine that mediates stem cell therapeutic effects.

Thus, in some embodiments fibroblasts produce enhanced production of one or more cytokines, such as HGF, following exposure to an effective amount of Poly (I:C), when compared to fibroblasts that were not exposed to the cytokine(s), such as HGF. Therapeutic properties of HGF include: stimulation of liver regeneration, stimulation of renal tubular epithelial cell proliferation, enhancement of recovery of renal function after injury, stimulation of keratinocyte growth, stimulation of angiogenesis, inhibition of cancer cell proliferation, stimulation of hematopoiesis, enhances B cell activity, stimulation of bronchial epithelial cell growth, stimulation of type 2 alveolar epithelial cells, inhibitory of epithelial cell apoptosis, stimulation of lung healing, reduction of pulmonary fibrosis, enhancement of pancreatic regeneration, promotes survival of neurons, promotes growth of axons, activation of muscle satellite cells, accelerates reconstitution of intestinal epithelial cells, accelerate post cardiac infarct recovery, suppresses cardiomyopathy, inhibits autoimmune myocarditis, reduces endothelial cell injury, reduces graft versus host disease, reduction of stroke size and acceleration of recovery, suppression of neuronal death, increases brain hypoperfusion, inhibits progression of neurodegenerative diseases, generates more oligodendrocytes, improves efficacy of islet transplantation, restoration of hearing impairment, stimulation of neuronal migration [116], suppression of inflammatory bowel disease, attenuates ischemia associated learning dysfunction, enhances synaptic plasticity, protects against blindness, stimulates production of interleukin 1 receptor antagonist, and so forth.

### REFERENCES

All publications mentioned in the specification are indicative of the level of those skilled in the art to which the invention pertains. All publications are herein incorporated by reference to the same extent as if each individual publication was specifically and individually indicated to be incorporated by reference.
1. Landen, N. X., D. Li, and M. Stahle, Transition from inflammation to proliferation: a critical step during wound healing. Cell Mol Life Sci, 2016. 73(20): p. 3861-85.
2. Reinke, J. M. and H. Sorg, Wound repair and regeneration. Eur Surg Res, 2012. 49(1): p. 35-43.
3. Ho, S., H. Marcal, and L. J. Foster, Towards scarless wound healing: a comparison of protein expression between human, adult and foetal fibroblasts. Biomed Res Int, 2014. 2014: p. 676493.
4. Adzick, N. S. and M. T. Longaker, Scarless fetal healing. Therapeutic implications. Ann Surg, 1992. 215(1): p. 3-7.
5. Zhang, X. Y., et al., Lentiviral vectors for sustained transgene expression in human bone marrow-derived stromal cells. Mol Ther, 2002. 5(5 Pt 1): p. 555-65.
6. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.
7. Worsham, D. N., et al., In vivo gene transfer into adult stem cells in unconditioned mice by in situ delivery of a lentiviral vector. Mol Ther, 2006. 14(4): p. 514-24.
8. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.
9. Kallifatidis, G., et al., Improved lentiviral transduction of human mesenchymal stem cells for therapeutic intervention in pancreatic cancer. Cancer Gene Ther, 2008. 15(4): p. 231-40.
10. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.
11. McGinley, L., et al., Lentiviral vector mediated modification of mesenchymal stem cells & enhanced survival in an in vitro model of ischaemia. Stem Cell Res Ther, 2011. 2(2): p. 12.
12. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.
13. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.
14. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.
15. Yu, Y. S., et al., AKT-modified autologous intracoronary mesenchymal stem cells prevent remodeling and repair in swine infarcted myocardium. Chin Med J (Engl), 2010. 123(13): p. 1702-8.
16. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.
17. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.
18. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.
19. Sage, E. K., et al., Systemic but not topical TRAIL-expressing mesenchymal stem cells reduce tumour growth in malignant mesothelioma. Thorax, 2014. 69(7): p. 638-47.
20. Lian, W. S., et al., In vivo therapy of myocardial infarction with mesenchymal stem cells modified with prostaglandin I synthase gene improves cardiac performance in mice. Life Sci, 2011. 88(9-10): p. 455-64.
21. Maijenburg, M. W., et al., Nuclear receptors Nur77 and Nurr1 modulate mesenchymal stromal cell migration. Stem Cells Dev, 2012. 21(2): p. 228-38.
22. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.
23. Zou, D., et al., In vitro study of enhanced osteogenesis induced by HIF-1alpha-transduced bone marrow stem cells. Cell Prolif, 2011. 44(3): p. 234-43.
24. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.
25. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.
26. Yang, H. M., et al., Enhancement of the immunosuppressive effect of human adipose tissue-derived mesenchymal stromal cells through HLA-G1 expression. Cytotherapy, 2012. 14(1): p. 70-9.
27. Liang, X. J., et al., Differentiation of human umbilical cord mesenchymal stem cells into hepatocyte-like cells by hTERT gene transfection in vitro. Cell Biol Int, 2012. 36(2): p. 215-21.
28. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.
29. Jaganathan, B. G. and D. Bonnet, Human mesenchymal stromal cells senesce with exogenous OCT4. Cytotherapy, 2012. 14(9): p. 1054-63.
30. Han, S. H., et al., Effect of ectopic OCT4 expression on canine adipose tissue-derived mesenchymal stem cell proliferation. Cell Biol Int, 2014. 38(10): p. 1163-73.
31. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.
32. Kearns-Jonker, M., et al., Genetically Engineered Mesenchymal Stem Cells Influence Gene Expression in Donor Cardiomyocytes and the Recipient Heart. J Stem Cell Res Ther, 2012. Sl.
33. Ma, G. L., et al., [Study of inhibiting and killing effects of transgenic LIGHT human umbilical cord blood mesenchymal stem cells on stomach cancer]. Zhonghua Wei Chang Wal Ke Za Zhi, 2012. 15(11): p. 1178-81.
34. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.
35. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.
36. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
37. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.
38. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.
39. Cao, Z., et al., Protective effects of mesenchymal stem cells with CXCR4 up-regulation in a rat renal transplantation model. PLoS One, 2013. 8(12): p. e82949.
40. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.
41. Zhu, Y., et al., Mesenchymal stem cell-based NK4 gene therapy in nude mice bearing gastric cancer xenografts. Drug Des Devel Ther, 2014. 8: p. 2449-62.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of enhancing one or more therapeutic properties of a fibroblast population comprising the step of treating the fibroblast population with an effective amount of exogenous RNA sufficient to enhance the one or more therapeutic properties of the fibroblast population.

2. The method of claim 1, wherein said RNA is double stranded RNA.

3. The method of claim 1, wherein said double stranded RNA is polyinosine-polycytidylic acid (Poly (I:C)).

4. The method of claim 1, wherein said double stranded RNA is polyinosine-polycytidylic acid stabilized with Polylysine and Carboxymethylcellulose (Poly ICLC).

5. The method of claim 1, wherein said therapeutic properties of said fibroblast population comprises production of one or more angiogenic factors.

6. The method of claim 1, wherein said therapeutic properties of said fibroblast population comprises production of one or more regenerative factors.

7. The method of claim 1, wherein said therapeutic properties of said fibroblast population comprises migratory activity towards one or more injury-associated signals.

8. The method of claim 1, wherein said therapeutic properties of said fibroblast population comprises reduction of apoptosis.

9. The method of claim 1, wherein said fibroblast population is derived from a source selected from the group of tissues consisting of a) adipose; b) dermal; c) placental; d) hair follicle; e) keloid; f) bone marrow; g) peripheral blood; h) umbilical cord; i) foreskin; j) a combination thereof.

10. The method of claim 1, further comprising the step of producing the fibroblast population.

11. The method of claim 10, wherein the producing step comprises inducing activation of toll like receptor 3 through contact with at least one ligand capable of inducing an interferon response in said fibroblast population.

12. The method of claim 1, wherein the method comprises the step of delivering a therapeutically effective amount of the fibroblast population to an individual that has a medical condition or is at risk for having a medical condition.

13. The method of claim 1, wherein the treated fibroblast population comprises an enhanced production of one or more cytokines compared to an untreated fibroblast population.

14. The method of claim 13, wherein the cytokine is HGF.

15. A method of treating one or more medical conditions in an individual, comprising the step of providing to the individual an effective amount of fibroblasts that have been exposed to an effective amount of exogenous RNA.

16. The method of claim 15, wherein said RNA is double stranded RNA.

17. The method of claim 15, wherein said double stranded RNA is polyinosine-polycytidylic acid (Poly (I:C)).

18. The method of claim 15, wherein said double stranded RNA is polyinosine-polycytidylic acid stabilized with Polylysine and Carboxymethylcellulose (Poly ICLC).

19. The method of claim 15, wherein the medical condition is one or more inflammatory conditions, one or more neurodegenerative conditions, cancer, an injury, or a combination thereof.
